(19) United States
(12) Patent Application Publication
(10) Pub. No.: US 2010/0137839 A1
(43) Pub. Date: Jun. 3, 2010

(54) Blind End Catheter Guide

(75) Inventor: Vincent G. Copa, Minnetonka, MN (US)

Correspondence Address: AMS RESEARCH CORPORATION, 10700 BREN ROAD WEST, MINNETONKA, MN, 55343, US

(73) Assignee: AMS RESEARCH CORPORATION, Minnetonka, MN (US)

(21) Appl. No.: 12/628,641

(22) Filed: Dec. 1, 2009

Related U.S. Application Data
(60) Provisional application No. 61/118,758, filed on Dec. 1, 2008.

(51) Int. Cl. A61M 25/16; B23P 17/04; B32B 37/16

(52) U.S. Cl. 604/533; 29/282; 156/294

(57) ABSTRACT

A catheter guide for assisting individuals in affixing catheter tips on blind-end catheters. More specifically, the catheter guide includes a mounting ring for receiving a catheter tip. The catheter guide further includes at least two integrated prongs extending from the mounting ring for defining a receptacle adapted to receive and aligning the blind end catheter with the catheter tip. The catheter guide maintains alignment of the blind end catheter and catheter tip while a bonding agent sets up. The catheter guide also includes an integrated tapering section extending from the integrated prongs to the mounting ring thereby eliminating the need for a separate tapering assembly step.

### PRIORITY CLAIM

The present application claims priority to U.S. Provisional Application Ser. No. 61/118,758, filed Dec. 1, 2008, and entitled “BLIND END CATHETER GUIDE”, which is herein incorporated by reference in its entirety.

### FIELD OF THE DISCLOSURE

The invention relates generally to a catheter guide for assisting individuals in affixing catheter tips on blind end catheters. More specifically, the present invention relates to a catheter guide mountable on a catheter tip providing a receptacle capable of aligning the blind end of a catheter body with the end of the catheter tip and having an integrated taper to simplify the installation process.

### BACKGROUND OF THE DISCLOSURE

A medical catheter is essentially a long lumen inserted into a body cavity, such as, for example, an individual's urinary tract, The medical catheter is capable of navigating the operational end of the catheter to a specific location within the body cavity. Typically, a treatment apparatus is affixed to the operational end of the catheter for providing a medical treatment to the body cavity or surrounding tissue. For example, the TMX-3000 OFFICE THERMO THERAPY SYSTEM produced by American Medical Systems of Minnetonka, Minn. includes a catheter having microwave antenna mounted at the operational end of the catheter to provide targeted microwave energy internally to a specific tissue site rather than transmitting the microwave energy through the patient's skin and other tissue to reach the target tissue. The treatment apparatus may be manufactured as part of the operational end of the catheter or manufactured separately and affixed to the operational end of the catheter. The treatment apparatus is often incapable of navigating unassisted through the body cavity to reach the target tissue due to the size, shape or rigidity of the treatment apparatus. A catheter tip may be affixed to the end of a catheter having an integrated treatment apparatus or affixed to a separately manufactured integrated treatment apparatus and affixed to the operational end of the catheter as whole unit. The catheter tip typically comprises a navigational portion that is smaller in diameter, softer or more flexible than the integrated treatment apparatus to ease the movement of the catheter through the body cavity and may perform other functions to assist in certain treatments.

A catheter tip with only the navigational portion or having both the navigational portion and the integrated treatment apparatus is typically affixed to the end of catheter by aligning the ends of the tip with the end of the catheter and bonding the ends together with a bonding agent such as adhesive or plasticizer. For the purposes of this disclosure, a “catheter tip” refers to both tips having only a navigational portion and catheters having both navigational portion and an integrated treatment apparatus. The alignment of the catheter body and the catheter tip are complicated by the discrete diameter of the catheter body and the even narrower catheter tip. Open ended catheters have an exposed catheter lumen that may be sufficiently large in diameter to receive a portion of the catheter tip into the catheter lumen, as disclosed in U.S. Pat. No. 5,017,259 to Kohsai, the disclosure of which is herein incorporated by reference in its entirety. Inserting a portion of the catheter tip into the open lumen of the catheter body simplifies the step of aligning the catheter tip and the catheter. In contrast, closed ended or “blind ended” catheters are closed at the end preventing the catheter tip from being inserted into the catheter lumen. As a result, the catheter tip and the blind ended catheter must be aligned manually.

In addition to simply aligning the tip and the blind ended catheter, the alignment must be maintained while the bonding agent is applied and sets. Unlike open ended catheters where the catheter tip is retained within the catheter lumen, the alignment of the catheter tips and ends of blind ended catheters must be manually maintained until the bonding agent sets.

An approach to affixing catheter tips to blind ended catheters is bonding a plurality of cut tubing supports to the end of the catheter tip. The plurality of cut tubing supports is oriented to extend out from the end of the catheter tip to define a receptacle into which the blind end of the catheter can be received and retained while the catheter tip and the catheter body are bonded together. However, the added difficulty of individually bonding a plurality of cut tubing supports and correctly orientating the plurality of cut tubing supports increases the difficulty of properly affixing the catheter tip to the catheter.

In addition to aligning and bonding the catheter tip and the catheter, the process of affixing a catheter tip to a catheter typically includes a tapering process. The catheter tip, treatment apparatus and catheter can all be of different diameters leaving edges or surfaces that may catch on the walls of the body cavity causing the parts of the catheter to separate or possibly damaging the tissue of the body cavity. Similarly, the plurality of cut tubing supports can also have exposed edges or surfaces that can cause the cut tubing to catch on the walls of the body cavity and separate from the catheter. The tapering process typically involves covering the exposed edges or cut tubing ends with an angled or tapered element that is bonded to the catheter or the catheter tip or by forming an angled or tapered portion on the catheter body and catheter tip with adhesive or plasticizer. In either approach, the tapering process adds additional complex processing steps increasing the likelihood that the catheter tip will be improperly affixed. Consequently, there is a need for an apparatus and a method for affixing catheter tips on blind end catheters that simplifies the attachment process by assisting persons in aligning the catheter tip with the catheter body and eliminating the complex steps of affixing cut tubing supports and tapering.

### SUMMARY OF THE DISCLOSURE

Representative embodiments the present invention provide a catheter guide for aiding individuals in affixing catheter tips on blind end catheters. More specifically, the present invention comprises a catheter guide adapted to be mountable on the catheter tip to be affixed. The catheter guide comprises integrated prongs for defining a receptacle capable of receiving and aligning the catheter.

In one representative embodiment, a catheter guide can comprise a mounting ring and at least two integrated prongs defining a receptacle for receiving an end of a blind-end catheter. The mounting ring defines a lumen adapted to receive the end of the blind-end catheter tip. The integrated prongs further comprise a mounting end and a receptacle end. The mounting end of the integrated prongs are affixed to the mounting ring such that the receptacle end of each integrated prong extends outwardly in the same direction from the end of the catheter tip. The at least two integrated prongs define a receptacle capable of receiving and holding the end of a catheter such that the end of the catheter is in operational contact with the end of the catheter tip. The mounting end of the integrated prong can further comprise an integrated tapered portion. The integrated portion is angled such that prongs do not catch on the walls of the body cavity as the catheter is navigated through the body cavity.

Another embodiment of a catheter guide can be molded to form a single piece of rigid material such that the affixing process only requires fitting the end of the tip into the lumen defined by the mounting ring and inserting the end of the catheter into the receptacle defined by the integrated prongs. The catheter guide eliminates the assembly steps of individually bonding cut tubing onto the tip and the catheter. Similarly, the catheter guide also eliminates the tapering step as a tapering portion is integrated into the catheter guide.

The above summary of the invention is not intended to describe each illustrated embodiment or every implementation of the present invention. The Figures and the Detailed Description that follow more particularly exemplify these embodiments.

### BRIEF DESCRIPTION THE FIGURES

The invention may be more completely understood in consideration of the following detailed description of various embodiments of the invention in connection with the accompanying drawings, in which:

FIG. 1 is a side view of a catheter system including a catheter, treatment apparatus and tip.

FIG. 2 is a side view of a prior art apparatus for affixing a tip to catheter.

FIG. 3 is side view of a blind end catheter guide according to an embodiment of the present invention for affixing a catheter tip having an integrated treatment apparatus to a catheter.

FIG. 4 is a side view of a blind end catheter guide according to an embodiment of the present invention for affixing a catheter tip to a catheter having an integrated treatment apparatus.

FIG. 5 is a side view an embodiment of a blind end catheter guide.

FIG. 6 is a front view of the blind end catheter guide of FIG. 5.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention as defined by the appended claims.

### DETAILED DESCRIPTION OF THE FIGURES

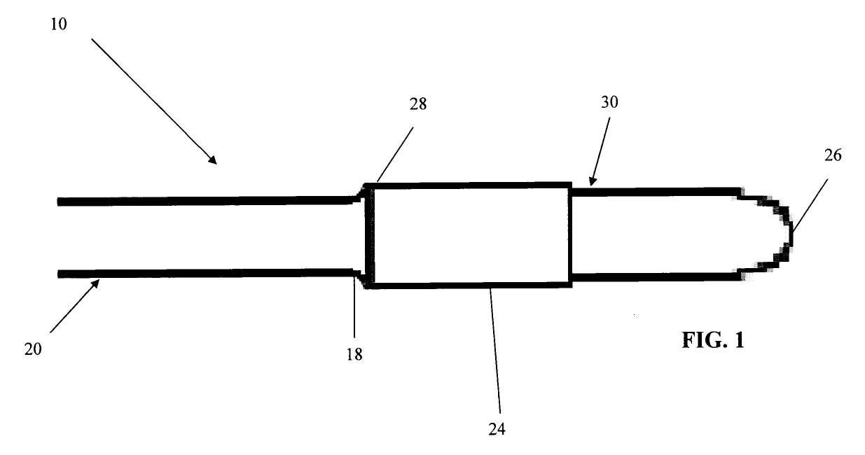

As illustrated in FIG. 1, a catheter system 10 generally comprises a blind-end catheter 20 and a catheter tip 30. As depicted, the catheter tip 30 further comprises an integrated treatment apparatus 24. The catheter tip 30 is defined by a guide end 26 and a mounting end 28. Blind-end catheter 20 further includes a blind end 18. The blind end 18 of the blind-end catheter 20 is bonded to the mounting end 28 of the catheter tip 30 to assemble the catheter system 10.

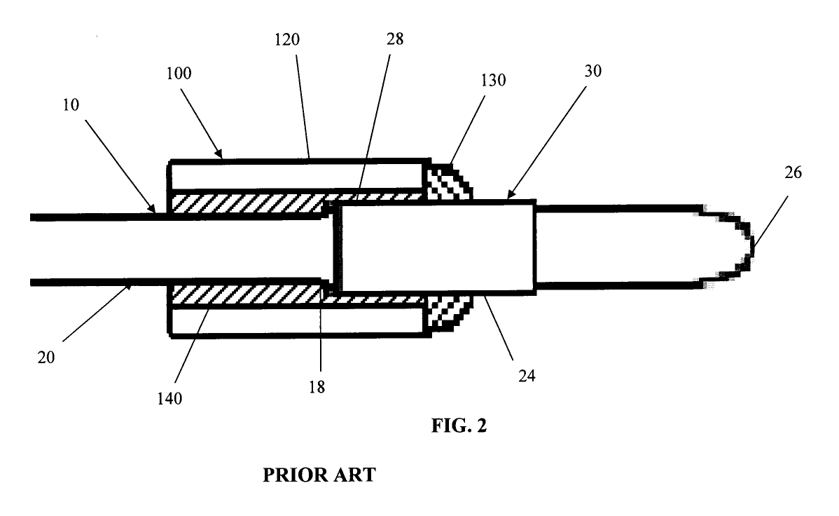

Referring to FIG. 2, a prior art assembly system 100 for aligning and affixing a catheter tip 30 to a blind-end catheter 20 comprises cut tubing supports 120. The cut tubing supports 120 are bonded with a bonding agent 140 to the mounting end 28 of the catheter tip 30 such that the cut tubing supports 120 extend outwardly from the mounting end 28 of the catheter tip 30. The cut tubing supports 120 are also bonded to the blind end 18 to hold the blind end 18 and mounting end 28 of catheter tip 30 in operational contact until the bonding agent 140 sets. The assembly system 100 further comprises tapering 130 disposed on that catheter tip 30 at the ends of the cut tubing supports 120. The assembly system 100 requires a number of additional assembly steps that rely heavily on the skill of the assembling individual to be properly performed. These additional assembly steps can include bonding the cut tubing supports 120 to the catheter tip 30 and tapering 130 disposed on the catheter tip 30.

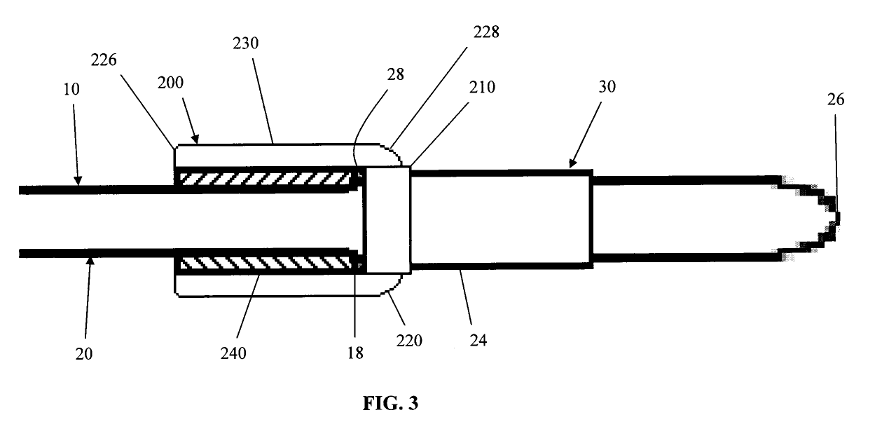

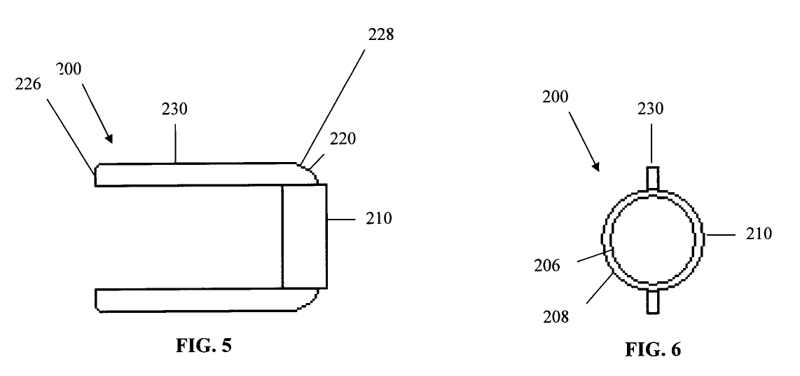

In contrast, FIGS. 3 and 5-6 illustrate an embodiment of a blind-end catheter guide 200 according to the present disclosure that comprises a mounting ring 210 defining a lumen 206 adapted to receive the mounting end 18 of the catheter tip 30 and at least two integrated prongs 230. The integrated prongs 230 each further comprise a mounting end 238 and a receptacle end 236. The mounting ends 238 of the integrated prongs 230 are adjoined to exterior 208 of the mounting ring 210 such that the receptacle ends 236 extend outwardly and define a receptacle for receiving the catheter end 18. The blind-end catheter guide 200 can be molded from a single piece of rigid material to eliminate the additional processing step of bonding the integrated prongs 230 to the mounting ring 210.

A catheter tip 30 is affixed to a catheter 20 by fitting the mounting end 18 of the catheter tip 30 into the lumen 206 of the mounting ring 210 such that integrated prongs 230 extend outwardly from mounting end 18 of the catheter tip 30. The mounting ring 210 can be bonded to the catheter tip 30 or held in place statically. The catheter end 18 is inserted into the receptacle defined by the outwardly extended integrated prongs 230 until the catheter end 18 is in operational contact with the mounting end 28 of the catheter tip 30. The integrated prongs 230 are bonded 240 to the catheter end 18 to secure the mounting end 28 of the catheter tip 30 to the catheter end 18

The integrated prongs 230 further comprise a tapered portion 228 at the mounting end 238 of the integrated prongs 230. Tapered portion 238 eliminates the additional assembly step of forming a tapered portion or bonding a tapered element onto the blind-end catheter guide 200.

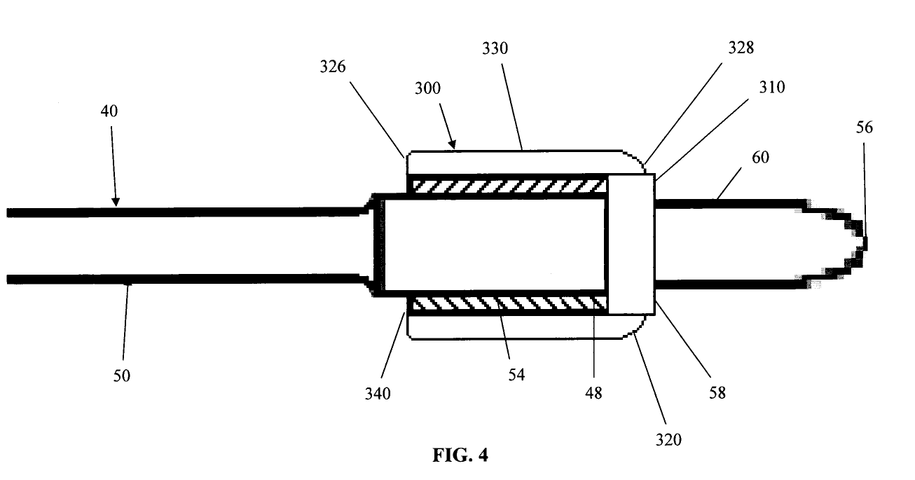

Alternatively, a catheter system 40 comprises a catheter 50 and a catheter tip 60 as illustrated in FIG. 4. Catheter 50 further comprises an integrated treatment apparatus 54. The catheter tip 30 is defined by a guide end 56 and a mounting end 58. The integrated treatment apparatus 54 comprises a blind-end 58 for the catheter 50. The catheter 50 can comprise an open lumen catheter 50 which closed off by the treatment apparatus 54 or a blind end catheter 50. In operation, the blind-end 58 of the treatment apparatus 54 is bonded to the mounting end 58 of the catheter tip 30.

A representative embodiment of a catheter guide 300 adapted to interface with the alternative catheter system 40 comprises a mounting ring 310 adapted to receive the mounting end 58 of the catheter tip 30 and at least two integrated prongs 330. The interior lumen defined by the mounting ring 310 and the diameter of the mounting ring 310 can be adjusted depending on the size of the catheter, treatment apparatus or tip. The catheter tip 60 is affixed to the treatment apparatus 54 and the catheter 40 by fitting the mounting end 58 of the catheter tip 30 into the mounting ring 310 such that integrated prongs 330 extend outwardly from the mounting end 58 of the catheter tip 30 and define a receptacle for the treatment apparatus 54. The treatment apparatus 54 is inserted into the receptacle for the integrated prongs 330 and bonded to the integrated prongs 330 with a bonding agent 340.

Although specific examples have been illustrated and described herein, it will be appreciated by those of ordinary skill in the art that any arrangement calculated to achieve the same purpose could be substituted for the specific example shown. This application is intended to cover adaptations or variations of the present subject matter. Therefore, it is intended that the invention be defined by the attached claims and their legal equivalents.

What is claimed:

1. A catheter guide for affixing catheter tips onto catheters comprising:
a mounting ring defining a lumen adapted to receive a portion of the catheter tip; and
at least two integrated prongs each having a first and second end, wherein the first end of each integrated prong is affixed to the mounting ring such that the integrated prongs extend outwardly from the mounting ring wherein the second ends define a receptacle configured to receive a catheter end.

2. The catheter guide of claim 1, wherein the mounting ring and integrated prongs are molded to form a single rigid piece.

3. The catheter guide of claim 1, wherein each integrated prong includes a tapered portion integrally molded at the first end.

4. A method of affixing catheter tips onto catheters comprising:
providing a catheter guide including a mounting ring defining a lumen for receiving a portion of the catheter tip and at least two integrated prongs, each integrated prong having a first and second end, wherein the first end of each integrated prong is affixed to the mounting ring such that the second ends define a receptacle capable of receiving a catheter;
inserting an end portion of the catheter tip into the lumen defined by the mounting ring;
inserting a catheter end of a catheter into the receptacle defined by the second ends until the catheter end is in fluid communication with the end portion of the catheter; and
bonding the catheter to the integrated prongs to secure the catheter to the catheter tip.

5. The method of claim 4, wherein the step of providing the catheter guide, further comprises:
molding the catheter guide such that the mounting ring and the integrated prongs comprise a single rigid piece.

6. The method of claim 5, wherein the step of molding the catheter guide, further comprises:
forming a tapered portion at the first ends of the integrated prongs, said tapered portion transitioning between the integrated prongs and the mounting ring.

7. The method of claim 4, wherein the step of bonding the catheter to the integrated prongs, further comprises:
retaining the catheter and catheter tip in fluid communication using the catheter guide as a bonding agents sets.

8. The method of claim 4, wherein the step of inserting the end portion of the catheter tip into the lumen defined by the mounting ring, further comprises;
bonding the catheter tip to the mounting ring.

9. The method of claim 8, wherein bonding the catheter tip to the mounting ring, further comprises:
retaining the catheter tip within the lumen defined by the mounting ring as a bonding agents sets.

10. A catheter system, comprising:
a catheter having a blind end;
a catheter tip defined by a mounting end and a guide end; and
a catheter guide for operably coupling the blind end to the mounting end, the catheter guide including a mounting ring defining a lumen adapted to receive the mounting end of the catheter tip, the catheter guide further including at least two integrated prongs having first and second ends, wherein the first end of each integrated prong is affixed to the mounting ring such that the second ends define a receptacle configured to receive the blind end.

11. The catheter system of claim 10, wherein the mounting ring and integrated prongs are molded such that the catheter guide is a single rigid piece.

12. The catheter system of claim 10, wherein each integrated prong includes a tapered portion integrally molded at the first end, said tapered portion extending continuously between the first end and the mounting ring.

13. The catheter system of claim 10, further comprising:
a bonding agent securing the integrated prongs to the catheter.

14. The catheter system of claim 13, further comprising:
a bonding agent securing the catheter tip within the mounting ring.
